(19) United States
(12) Patent Application Publication
(10) Pub. No.: US 2014/0121561 A1
(43) Pub. Date: May 1, 2014

(54) Specimen Collection Device

(71) Applicant: Victor Chacchia, Carrollton, TX (US)

(72) Inventor: Victor Chacchia, Carrollton, TX (US)

(21) Appl. No.: 13/666,472

(22) Filed: Nov. 1, 2012

(51) Int. Cl. A61B 5/20

(52) U.S. Cl. 600/574

(57) ABSTRACT

The present invention is a specimen collection device. The specimen collection device is designed to provide a convenient and sanitary way to collect a specimen. The device is comprised of an upper portion with an opening, a lower portion with another opening, a handle and a securing mechanism. The oval shaped contour of the upper portion of the device conforms to female body. The handle at the exterior edge of the upper portion includes grips on the top and bottom surfaces of the handle to prevent the device from slipping from the users hands. The device includes a securing mechanism such as threads on the device that interlock with the threads on the opening a storage. The securing mechanism firmly connects the device to the storage eliminating leakage and loss due to misdirection.

### FIELD OF THE INVENTION

The present invention relates to specimen collection, and in particular urine sample collection devices.

### BACKGROUND OF THE INVENTION

Urine samples are collected in the medical field for various purposes. Urine samples are occasionally collected to test for the presence or absence of substances in the body as in the case of drug tests. Urine samples have also been collected to screen for specific medical conditions like diabetes or kidney stones. Urine samples are typically collected at the office of a physician or other medical professional. The patient is provided with a cup and a private space where the patient can fill the container with a urine specimen.

While a male can usually direct the urine flow into a urine container with relative ease, for a female, this procedure may be burdensome. A female generally has to squat or sit to collect a urine specimen. Not only it is hard to balance while squatting, but a female has limited visibility to place the urine container beneath the flow of urine or direct the urine flow. Thus, a female usually has more difficulties accurately directing the flow of her urine to the container without spillage or back splash. The process can be awkward and unsanitary in cases of splashing and spillage. For handicapped or disabled persons, the difficulty in collecting urine may be greater because they may not be able to squat, reach between their legs or position the urine container properly to collect the urine.

To deal with these difficulties, some patients use funnels with the urine container. The funnel eliminates some difficulties for a female by providing a larger collection area for the specimen. However, this solution is not completely satisfactory. The round top of the funnel does not mimic the anatomy of a female, therefore lessening the coverage of the female genitalia and the comfort to the user. In addition, the user of the funnel faces the inconvenience of manually holding the funnel and the urine container together while collecting the urine. If the funnel is attached to the container by snapping on or exterior clasps, urine may spill or splash while the user attempts to remove the funnel from the container. Furthermore, the user lacks any means to balance the funnel with the container while squatting in an awkward position.

It is desirable to provide an improved device for collecting urine that is convenient to use. The present device conforms to the female anatomy creating a greater coverage of the female genitalia. In addition, the specimen collection device includes a screwing mechanism to securely attach the device to a collection container which allows the specimen collection device to be removed with substantially decreased risk of specimen spillage. Furthermore, a handle is attached to the device for convenient transfer and handling.

### SUMMARY OF THE INVENTION

The present invention is a specimen collection device. The specimen collection device is comprised of a funnel like component, a handle, and a securing mechanism. The specimen collection device includes two openings. The first opening is located at the upper portion of the specimen collection device and the second opening is located at the lower portion of the specimen collection device. The opening at the upper portion of the specimen collection device is preferably oblong. The lip of the specimen collection device surrounds the boundary of the first opening. From the lip of the specimen collection device, the diameter of the upper portion of the specimen collection device uniformly decreases to the lower portion of the specimen collection device. The lower portion of the device has a uniform diameter substantially equivalent to or slightly larger than the diameter of a container or the opening of the container. The opening at the lower portion of the specimen collection device guides the specimen flow to the container or another device attached to the lower portion of the specimen collection device. In this embodiment, the specimen is urine but the specimen may be any of a variety of human waste products.

In use, the specimen collection device is positioned such that the upper portion, with the wider diameter, its between the legs of the users, while creating a larger surface area to cover female genitalia and therefore prevent spillage or loss due to misdirection. The lip at the upper portion of the specimen collection device, may be smooth to eliminate irritation or abrasion to the users' skin. The inner surface of the specimen device is preferably made of a material that does not absorb liquid such as plastic, vinyl or waxed coating.

To aid the user in holding the specimen collection device, a handle is located on the exterior edge of the lip. The handle includes grips on both the top surface and bottom surface of the handle to prevent the specimen collection device slipping from the users' hands. The thumb of the user may be positioned on the top surface of the handle while the index finger of the user may be positioned on the bottom surface of the handle. The grips on the surfaces of the handle prevent the hands from slipping while holding the specimen collection device. In addition, the handle allows the users to hold the device without touching the inner or outer surface areas of the specimen collection device, which may have come into contact with urine flow. Also use of the handle allows the user to better balance the specimen collection device while squatting. By using the handle, the user does not have to reach as far to position the specimen collection device to collect the specimen. Moreover, using the handle, the user can also wash the device without touching the areas contaminated by urine. Alternatively, the opening at the lower portion of the specimen collection device may connect with the opening of a hose or container as long as the opening at the lower portion of the specimen collection device and the openings of the hose or the container are of similar sizes. A hose connected to the specimen collection device would be useful in collecting samples from disabled persons or other with limited mobility.

The lower portion of the specimen collection device includes a securing mechanism that firmly connects the specimen collection device to a container or hose. The securing mechanism may also prevent leakage of contents passing between the specimen collection device and the container or hose. The securing mechanism for the specimen collection device is preferably a screwing mechanism. The screwing mechanism allows the users to separate the specimen collection device and the urine container. The screwing mechanism is comprised of threads on the inner surface of the lower portion of the specimen collection device. The urine container should include outer threads that interlock with the inner threads of the specimen collection container to completely seal and connect the specimen collection device to the container. To use the specimen collection device with a hose, the threads or exterior surface of hose should be capable of screwing onto the interior surface of the lower portion of the specimen connection device. The securing mechanism eliminates the needs for the users to manually attach the container and the funnel, which may increase chances of spillage or leakage.

The present invention eliminates discomfort and inconvenience by providing a specimen collection device that accommodates to the female anatomy. Also the present invention limits splashing and spilling of specimen with a steady securing mechanism. In addition, the handle allows the user to better balance the specimen collection device when collecting the specimen.

### BRIEF DESCRIPTION OF THE DRAWINGS

For a more complete understanding, attached are drawings which show the system and method in various configurations.

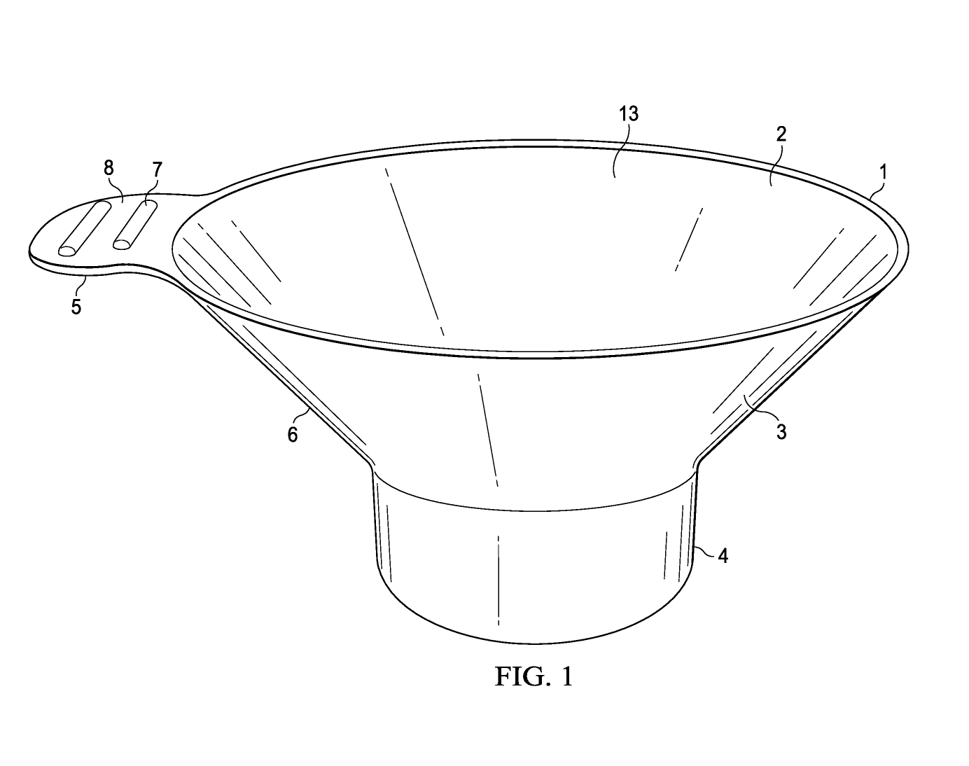

FIG. 1 is a view of a specimen collection device.
Ref. 1 is a lip;
Ref. 2 is an inner surface of the specimen collection device;
Ref. 3 is an outer surface of the specimen collection device;
Ref. 4 is a lower portion of the specimen collection device;
Ref. 5 is a handle;
Ref. 6 is an upper portion of the specimen collection device;
Ref. 7 is a grip on the top surface of the handle;
Ref. 8 is a top surface of the handle;
Ref. 13 is an opening at the upper portion of the specimen collection device.

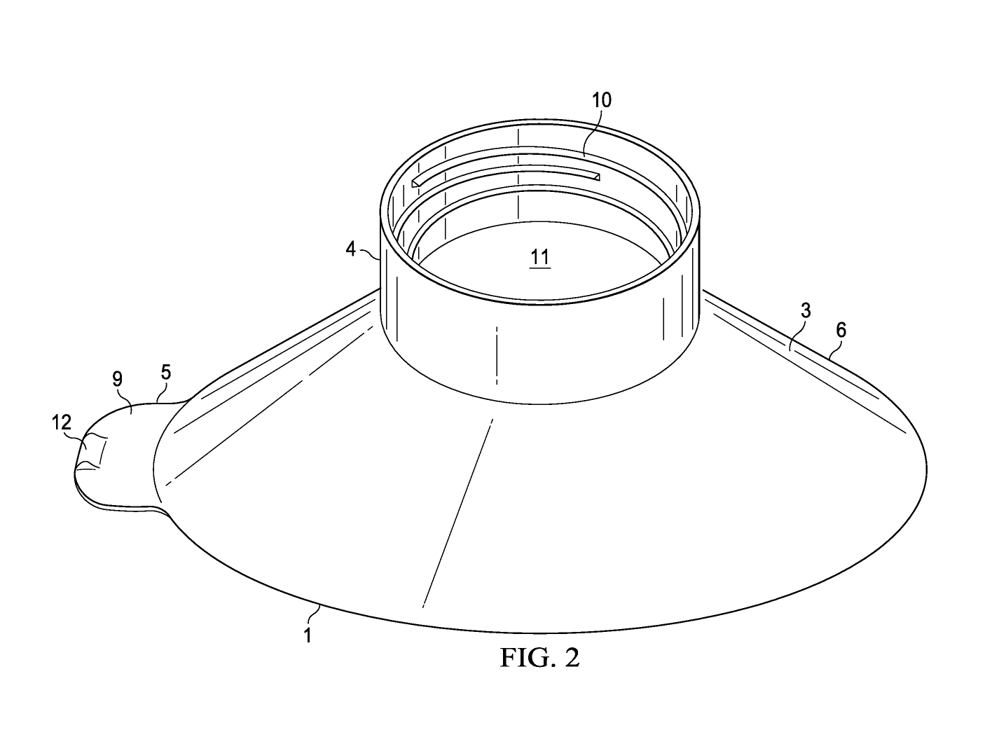

FIG. 2 is a view of a specimen collection device upside down.
Ref. 1 is a lip;
Ref. 3 is an outer surface of the specimen collection device;
Ref. 4 is a lower portion of the specimen collection device;
Ref. 5 is a handle;
Ref 6 is an upper portion of the specimen collection device;
Ref. 9 is the bottom surface of the handle;
Ref. 10 is threads;
Ref. 11 is the opening, at the lower portion of the specimen collection device;
Ref. 12 is a grip on the bottom surface of the handle;
Ref. 13 is an opening at the upper portion of the specimen collection device.

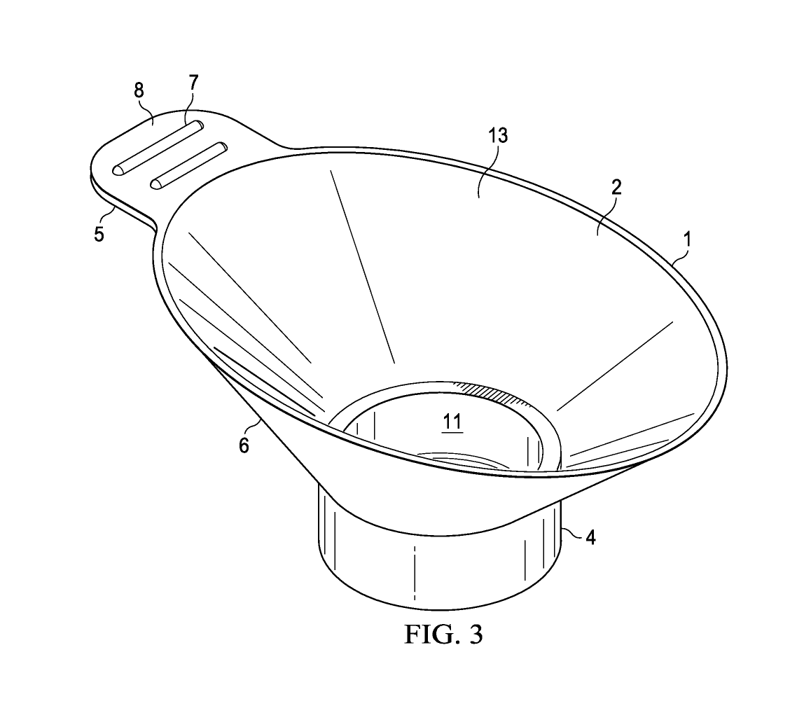

FIG. 3 is a side view of a specimen collection device upside down.
Ref. 1 is a lip;
Ref. 2 is an inner surface of the specimen collection device;
Ref. 5 is a handle;
Ref. 6 is an upper portion of the specimen collection device;
Ref. 7 is a grip on the top surface of the handle;
Ref. 8 is a top surface of the handle;
Ref. 11 is the opening at the lower portion of the specimen collection device;
Ref. 12 is a grip on the bottom surface of the handle.

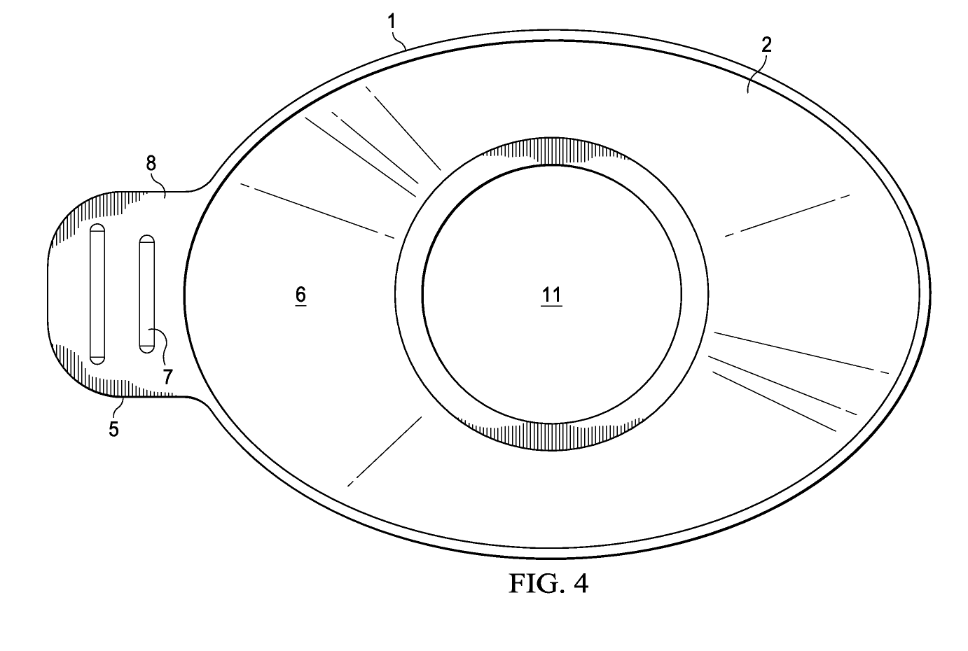

FIG. 4 is a top view of a specimen collection device.
Ref. 1 is a lip;
Ref. 2 is an inner surface of the specimen collection device;
Ref. 5 is a handle;
Ref. 6 is an upper portion of the specimen collection device;
Ref. 7 is a grip on the top surface of the handle;
Ref. 8 is a top surface of the handle;
Ref. 11 is the opening at the lower portion of the specimen collection device.

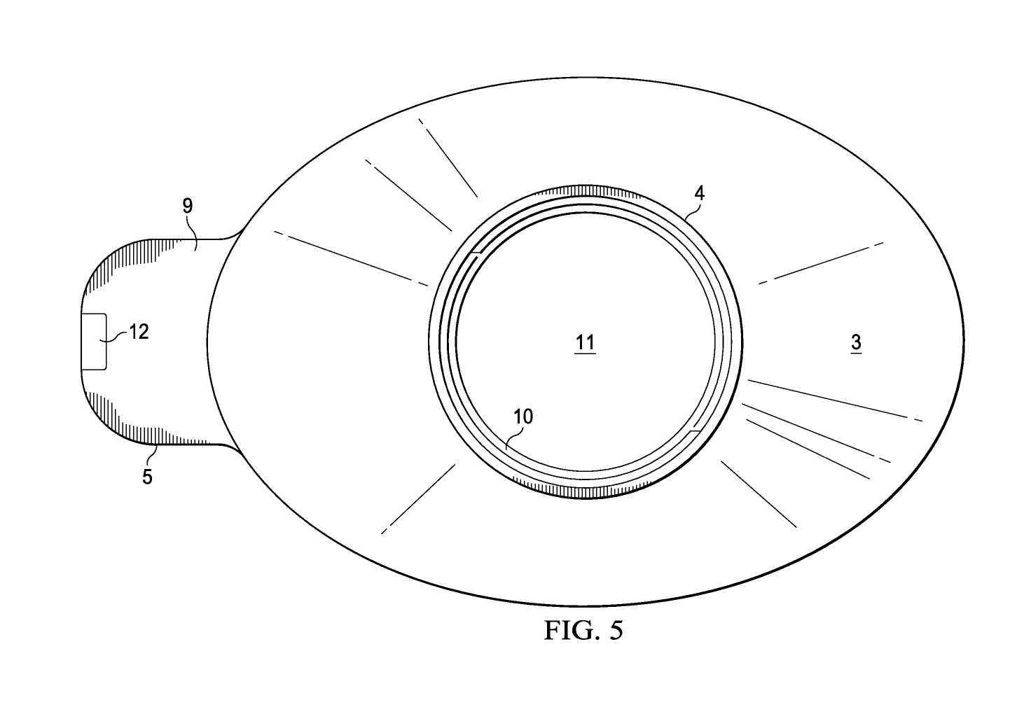

FIG. 5 is a bottom view of the specimen collection device.
Ref. 3 is an outer surface of the specimen collection device;
Ref. 4 is a lower portion of the specimen collection device;
Ref. 5 is a handle;
Ref. 9 is the back of the handle;
Ref. 10 is a thread;
Ref. 11 is the opening at the lower portion of the specimen collection device;
Ref. 12 is a grip on the bottom surface of the handle;
Ref. 13 is an opening at the upper portion of the specimen collection device.

### DETAILED DESCRIPTION OF THE PREFERRED EMBODIMENT

While the above description is of the preferred embodiment of the present invention, it should he appreciated that the invention may be modified, altered or varied without deviating from the scope and fair meaning of the following claims.

Referring generally to FIGS. 1-5, the specimen collection device is designed to collect specimens in a convenient way. The specimen collection device is comprised of a funnel like device, a handle 5, and a securing mechanism 10. The specimen collection device includes an upper portion 6 and a lower portion 4. The upper portion of the specimen collection device 6 includes an opening 13 with a boundary or a lip 1. The lower portion of the specimen collection device 4 also includes an opening 11 and threads 10. A handle 5 is located on the exterior edge of the lip 1. The handle 5 has grips 7 on the top surface of the handle 8 and an extra grip 12 on the bottom surface of the handle 9.

FIG. 1 is a view of the specimen collection device. The specimen collection device has an upper portion 6 and a lower portion 4. The upper portion of the specimen collection device 6 includes an opening 13 with a lip 1. In this embodiment, the opening 13 at the upper portion of the specimen collection 6 is an oval shape. When the specimen collection device is in use, the specimen collection device is positioned such that the opening 13 with the wider diameter fits between the legs of the user. The oval shape creates a larger coverage area for female genitalia to prevent spillage or loss due to misdirection. In addition, the oval shape fits more conveniently between legs of the user. The lip 1 is smooth to prevent, irritation or abrasion to the users' skin. The lower portion of the specimen collection device 4 includes the second opening 11 that may connect to the opening of the container, storage or hose and guide the urine to flow to the opening of the container or hose.

The user directs the specimen into the opening 13 of the upper portion of the specimen collection device 6, where the specimen hits the inner surface of the specimen collection device 2 and flows to the lower portion of the specimen collection device 4. The specimen collection device, especially the inner surface 2, is preferably made of a material that does not absorb liquid and is light weight such as plastic, vinyl or waxed coating. A handle 5 is located on the exterior edge of the lip 1. One or more grips 7 are located on the top surface of the handle 8.

FIG. 2 is a bottom side view of the specimen collection device. The diameter of the upper portion of the specimen collection device 6 uniformly decreases from the lip 1 to the top of the lower portion of the device 4. The opening at the lower portion of specimen collection device has a diameter substantially equivalent to or slightly larger than the diameter of the opening of the container, storage or hose. The lower portion of the specimen collection device 4 includes the opening 11 that attaches to the opening of the specimen container. The lower portion of the specimen collection device 4 includes a securing mechanism, such as threads 10. The user can screw the threads 10 of the specimen collection device onto the opening of the container, storage or hose. The container preferably includes outer threads that interlock with the inner threads of the specimen collection device 10. The screwing mechanism allows the user to conveniently connect the specimen collection device to the urine container. Also, the screwing mechanism completely seals and connects the specimen collection device and the container. Furthermore, the screwing mechanism eliminates the inconvenience of the manually holding the container and specimen collection device together.

The handle 5 includes another grip 12 on the bottom surface of the handle 9. Grips on both sides of the handle prevent the user from losing control of the specimen collection device. The thumb of the user is placed on the top surface of the handle and the index finger is placed on the bottom surface of the handle. The grips on the both sides of the handle prevent both fingers from slipping. In addition, the handle allows the user not to reach too far to position the specimen collection device and therefore the user can balance better while squatting.

FIG. 3 is the upper side view of the specimen collection device. The opening 13 at the upper portion of the specimen collection device 6 is a symmetrical oval in this embodiment. The urine hits the inner surface of the specimen collection device 2 and flows to the lower portion of the specimen collection device 4. The outer surface of the specimen collection device 6 may be made of similar material as the inner surface 2 of the specimen collection device. The handle 5 has one or more grips 7 located on the top surface of the handle 8. The lower portion of the specimen collection device 4 includes the opening 11 that directs the flow of the urine to a container or hose.

FIG. 4 is the top view of the specimen collection device. The opening 13 at the upper portion of the specimen collection device 6, is an oval shape and the opening 11 at the lower portion of the specimen collection device 4 is a circular shape. The opening 11 is attached to the opening of the container or hose. The upper portion 6 and the lower portion 4 of the specimen collection device are connected by the inner surface wall 2. The handle 5 with the grips 7 is located on the exterior edge of the lip 1.

FIG. 5 is a bottom view of the specimen collection device. The lower portion of the specimen collection device 4 includes an opening 11. Threads 10 are located in the inner surface of the lower portion 4 as a securing mechanism. The handle 5 includes a grip 12 on the bottom surface of the handle 9 for extra security and grip 7 on the top surface of the handle 8 for the users preventing from their fingers slipping while using the specimen collection device.

What is claimed is:

1. A specimen collection device, comprising:
a tube with an upper portion and a lower portion;
a securing mechanism; and
at least one grasping mechanism with at least one projection.

2. The specimen collection device of claim 1, wherein said upper portion of said tube has at least one opening.

3. The specimen collection device of claim 1, wherein said lower portion of said tube has at least one opening such that said opening of said upper portion and said opening of said lower portion form a channel through which a specimen may flow.

4. The specimen collection device of claim 1, wherein said tube is made of material selected from the group consisting of plastic, vinyl, cardboard, and waxed paper.

5. The specimen collection device of claim 1, wherein said grasping mechanism is located on an exterior edge of said upper portion of said tube.

6. The specimen collection device of claim 1, wherein said securing mechanism is located at said lower portion of said tube.

7. The specimen collection device of claim 1, wherein said securing mechanism is selected from the group consisting of screwing mechanism, snapping mechanism, and twist-lock mechanism.

8. (canceled)

9. (canceled)

10. The specimen collection device of claim 1, wherein said grasping mechanism includes at least one projection.

11. The specimen collection device of claim 1, wherein said opening of said upper portion is oblong.
